(19) United States
(12) Patent Application Publication
(10) Pub. No.: US 2007/0027049 A1
(43) Pub. Date: Feb. 1, 2007

(54) Anhydrous self-warming composition

(76) Inventor: Richard Rigg, Springfield Gardens, NY (US)

Correspondence Address: ANDREW F. YOUNG,;LACKENBAUGH SIEGEL, LLP, ONE CHASE ROAD, SCARSDALE, NY, 10583, US

(21) Appl. No.: 10/556,539

(22) PCT Filed: May 27, 2004
(86) PCT No.: PCT/US04/16985

§ 371 Date: Nov. 14, 2005

(30) Foreign Application Priority Data
May 27, 2003 (US) 60473220

(51) Int. Cl. A61K 8/00

(52) U.S. Cl. 510/130; 424/70.1

(57) ABSTRACT

An anhydrous self-warming composition includes a heat-generating powder having a reduced size generally coated with a combination of oils and/or waxes to minimize a later reaction rate when combined with water and enable an increased duration heating cycle. Additional components may optionally include alternative surfactants, skin benefit agents, fragrances, flavors, and colors.

### BACKGROUND OF THE INVENTION

1. Field of the Invention

The present invention relates to compositions of powders that release heat upon contacting with water, and that in alternative embodiments may be surface coated with, or optionally and alternatively combined with oils, waxes, surfactants, and other items. The invention, in alternative embodiments may also optionally relate to compositions and materials containing the powders in solid, liquid, viscous, or thixotropic forms, and methods for manufacturing the same.

2. Description of the Related Art

U.S. Pat. No. 3,250,680 (Menkart et al.) discloses a liquid composition containing zeolite as a heat-generating material.

U.S. Pat. No. 4,159,316 discloses an anhydrous self-heating dentifrice containing zeolite. Other exothermic anhydrous compositions include U.S. Pat. Nos. 4,626,550 , 4,379,143, and 4,362,715.

WO 02/060407 (Mitsumatsu et al.) discloses a self-heating anhydrous liquid cosmetic composition, particularly for hair conditioning, comprising (1) inorganic heat-generating agents, (2) polyoxyalkylene derivatives, and (3) a liquid carrier. The Mitsumatsu et al. composition is in a liquid state unsuitable for incorporation into an anhydrous base and does not contain the mild foaming surfactants and other elements of the present invention.

U.S. Pat. No. 6,540,989 (Janchitraponvej) disclosed a self-warming anhydrous rinse-out liquid hair conditioner comprising glycol and conditioning materials. U.S. Pat. No. 6,540,989 (Janchitraponvej) does not contain the heat-generating inorganic salts of the present invention.

### SUMMARY OF THE INVENTION

This invention relates to compositions of powders that, in optional, additional, or alternative embodiments, may be surface coated with anhydrous oils and/or waxes and surfactants, and release heat upon contact with water. Further optional and alternative embodiments of the present invention relate to compositions containing surface coated powders.

Alternative and adaptive embodiments of the present invention may also relate to a method for preparing the compositions of powders and methods for preparing cosmetic products containing the same.

Further alternative and optional embodiments of the present invention may provide cosmetic products containing the compositions of powders produced by the method wherein the powders are maintained in a powdered, solid packed, or liquid or viscous mass or thixotropic form, or even optionally retained within a solid matrix such as soap or on and in an individual-use application cleansing pad enabling later combination with water to generate a self-warming cleaning product.

In one alternative aspect of the present invention, a self-warming anhydrous powder composition is provided. In other optional and alternative embodiments, a method is provided for preparing and incorporating the same into an anhydrous base, a cleansing product, or a cosmetic for cleansing and treating body parts, especially skin.

One optional method for manufacturing a product according to the invention includes the steps of:
(1) Coating of a heat-generating powder with oils and thickeners (waxes, clays etc.)
(2) Reduction of particle size of the heat generating powder, prior to, during, or after a coating, by known mechanical means to a predetermined size or distribution of sizes.
(3) Varying the particle size of the heat generating powder from between about 0.1 um (microns) to 2 mm (millimeters), and in one alternative embodiment from about 0.1 um to about 500 microns. Other preferred particle size distributions, including normal distributions, are suggested below.

In additional embodiments further steps may include the alternative combination of several additional items noted below, including surfactants, fragrances, and inert fillers.

Self-warning cleansing compositions or products according to the present invention may include optionally various compositions or products comprising selected ones of the items in the ranges noted below:
(1) A heat-generating coated inorganic powder from 1% to 80% (by weight);
(2) Oils and Thickeners (Waxes, elastomers, clays, and others) from about 0.1%-90% (by weight);
(3) Surfactant from about 1%-30% (by weight); and optionally selected
(4) Fragrances/Flavors from about 0.10% to 10% (by weight); and, further optionally in selected alternative embodiments
(5) Skin Benefit Agents from about 0.10% to about 30% (by weight); and, still further optionally in alternative embodiments
(6) Additional Inert Fillers and Thickeners from about 0.00% to about 25% (by weight).

The present invention relates to an anhydrous self-warming skin care composition includes a heat-generating powder having a reduced size generally coated with a combination of oils and/or waxes to minimize a later reaction rate when combined with water and enable an increased duration heating cycle. Additional components may optionally include alternative surfactants, skin benefit agents, fragrances, flavors, and coloring components. The present invention optionally includes a method and process for producing an anhydrous self-warming skin care composition and a product made by the method and process for producing.

The above, and other alternative and adaptive items, features, objects, and advantages of the present invention will become apparent from the following description read in conduction with the accompanying drawing.

### BRIEF DESCRIPTION OF THE DRAWINGS

FIG. 1 is a graphical representation of the durable temperature change sustaining a warmed state over time for representative materials containing 20% heating materials.

### DETAILED DESCRIPTION OF THE PREFERRED EMBODIMENTS

This invention relates to compositions of powders or particles, that in alternative embodiments may be surface coated with oils and or waxes, and that release heat upon contacting or mixing with water. Alternative embodiments of this invention further relate to compositions containing the surface coated powders and methods for manufacturing the same.

It is desirable to release heat from cosmetic cleansing products during application on body parts, especially on the face. The heat generated from the exothermic reaction with water warms the skin and is believed to promote blood circulation. This warming effect is further believed to enhance cleansing, a perfume smell, and treatment benefits. The warm-sensation also provides a consumers with a very pleasant feel that, signals that the product is really working.

Inorganic salts, such as calcium chloride and magnesium sulphate and others react with water in an exothermic reaction and generate heat. It is proposed that inorganic salts are safe and beneficial as heat-generating components in cosmetic compositions. It is further proposed that one of the essential attributes that consumers may demands from a warming premium cosmetic cleansing product is an extended or continuous warming during a reasonable use period, as opposed to an immediate warming (short term) and then (rapid) loss of warmth. It is also proposed that consumers desire a clean feel upon rinse-off.

As noted above, self-warming cleansing compositions or products according to the present invention may include various compositions or products comprising selected items in the ranges noted below:
(1) A heat-generating coated inorganic powder from 1% to 80% (by weight);
(2) Oils and Thickeners (Waxes, elastomers, clays, and others) from about 1%-90% (by weight);
(3) Surfactant from about 1%-50% (by weight); and optionally
(4) Fragrances/Flavors from about 0.01% to 10% (by weight); and, further optionally in selected alternative embodiments additional
(5) Skin Benefit Agents from about 0.01% to about 30% (by weight); and, still further optionally in alternative embodiments additional
(6) Inert Fillers and Thickeners from about 0.00% to about 25% (by weight).

It should be understood that the above items may be selected within the ranges discussed above (or in further different or preferred ranges discussed below), and may be excluded in some embodiments. For example, optional alternative embodiments may include selectively (or together) items (1-3) and/or (4) and/or (5) and/or (6) dependent upon the desired end product, the texture of a desired end product, manufacturing control needs, a desired warming effect, a skin treatment need (skin sensitivity) or other factor. It should be understood that, for example that a fragrance/flavor may be excluded, or inert fillers or thickeners/elastomers/surfactants may be excluded where there are sufficient thickeners or other elements to achieve a desired texture. It should also be understood, that as discussed later, where there are sufficient thickeners, inert fillers may not be needed, and that some components of the thickeners and inert fillers listed below may be similar.

In one possible alternative embodiment of the present invention, the (1) oil and thickener combination may advantageously comprise, (based on the total weight of composition), from 5 % to 80%, (2) surfactants containing at least one ionic surfactant from 1-30%; (3) from 5% to 50% of heat-generating agents; (4) from 0.1% to 20 % of skin benefit agents; and (5) from 0% to 20% of inert fillers; and (6) 0.10% to 2% fragrance/flavor.

One optional method for manufacturing a product according to the invention includes the steps of:
(1) Coating of a heat-generating powder with oils and waxes.
(2) Reduction of particle size of the heat generating powder, prior to, during, or after a coating, by known mechanical means to a predetermined size or selected distribution of sizes.
(3) Varying the particle size of the heat generating powder from between about 0.1 um (microns) to 2 mm (millimeters), and in one alternative embodiment from about 0.1 um to about 500 microns. Other preferred particle size distributions, including normal distributions, are discussed below.

As used, the term “anhydrous” implies the presence of no more than 10%, preferably no more than 5%, and most preferably no more than 1%, water of the whole composition.

Water of hydration is not considered as content of water for the purpose of the defining “anhydrous”, although it is generally preferred to minimize or eliminate hydrated water.

As used, the term “powder” is meant a generally dry solid which may contain a certain amount of liquid components being deposited into or absorbed into the resulting flowing and flow-able powder. All powder components used in this composition are advantageously and preferably solid powders; alternatively the final composition in which the powders are incorporated may, in alternative and optional embodiments, be a free flowing liquid or a viscous mass or cream or thixotropic gel of some type. It is also to be understood, that one application of the product produced herein may be a powder in combination with a fibered washing pad (skin cleansing pad) enabling a user to apply the product to the pad (or use a pre-loaded pad), moisten the pad and wash using the pad to cleanse the skin. Alternative, the product produced herein may be incorporated into a soap or other cleansing product allowing activation of the thermal reaction during use.

Oils and Thickeners:

The first component includes the oil and thickener (e.g., waxes and other components including elastomers) mixture containing, in an optional selected embodiment, at least one wax of melting point in excess of 20 degrees centigrade and one oil that has a HLB value ranging from preferably 1-16. Examples of waxes include, but are not limited, to natural waxes and derivatives of such waxes (derived from plants and animals) and synthetic waxes. Representative examples of waxes may also include: beeswax, carnauba wax ,candelilla wax, jojoba wax, olive wax, spermaceti wax, shellac wax, montan wax, lanolin wax, polyethylene wax, microcrystalline wax, ozokerite wax, ceresin wax, petroleum and petroleum wax and ester waxes. Further representative examples of waxes are those modified by esterification, ethoxylation, propoxylation and combinations thereof.

In one optional preferred embodiment, the waxes are ethoxylated and propoxylated polyethylenes. The most preferred waxes of this optional embodiment are those having C10 to C60, propoxylation from 1 to 40 moles, and ethoxylation from 1 to 40 moles.

Examples of possible alternative thickeners (in addition to the waxes noted above) are elastomers. Elastomers may be used alone as thickeners or fillers or in combination with the waxes. Examples of suitable elastomers include, but are not limited to: Kratons (a hydrocarbon based elastomer) and EPSQ (a powdered elastomer) both available from Grant Industries, Inc. Of 125 Main Avenue, Elmwood park, N.J. 07407. Further examples of suitable thickeners include, but are not limited to, clays such as bentonites, hectorites and laponites.

Examples of oils include, but not limited to, vegetable and plant oils, including canola, sunflower, safflower, soybean, castor, rice bran, corn, coconut, palm, macadamia nut, almond; and synthetic oils including mineral, isoparaffin, esters, and silicone oils.

Surfactants:

Another component includes a single surfactant or a mixture of surfactants (often surfactant powders or in other easily used forms (liquid)) containing generally, and in a preferred embodiment at least one ionic surfactant which includes anionic or amphoteric surfactants.

Examples of anionic surfactants include surfactants selected from these classes of surfactants: alkyl sulphates, alkyl ether sulphates, alkyl monoglyceride sulphates, alkyl monoglyceride sulphonates, alkylaryl sulphates, alkyl sulfosuccinates, alkyl ether sulfosuccinates, alkyl sulfosuccinamates, alkyl ethercarboxylates, alkylamidoethercarboxylates, alkyl succinates, fatty acyl taurates, fatty acyl isethionates, fatty acyl sarcosinates, fatty acyl glutamates, and alkyl phosphates. Alternative surfactants may include or be combined with foaming surfactants or foaming agents suitable for use in foaming skin cleansers or on skin cleaning fibrous pads when mixed with water. The foaming action provided surfactant use aids in exfoliation of skin cells and additional cleaning benefits allowing a crisp clean feel following wash-off by a user. While surfactant use is not required in each embodiment one optional preferred embodiment includes surfactants.

In selected embodiments, the preferred surfactants are those having C10 to C16 in the fatty acyl part of the surfactant. In other selected embodiments, the most preferred anionic surfactants are those of isethionates, taurates, sulfosuccinates, sulfosuccinamates, and phosphates with C10 to C16 fatty acyl radical, and mixtures thereof.

Examples of amphoteric surfactants include surfactants selected from these classes of surfactants: amphocarboxylates, alkyl betaines, amidoalkyl betaines, amphophsphates, phosphobetaines, pyrophosphobetaines, carboxyalkyl polyamines, and amidoalkyl sultaines. In selected embodiments, the preferred surfactants are those having C10 to C16 in their fatty acyl part.

In alternative embodiments, the surfactant may include suitable nonionic surfactants including alkyl polyglucoside having alkyl groups from C10 to C16, and optionally cocoamidopropyl amine oxide.

The surfactant portion, based on the total weight of the surfactant portion, preferably contain from 20% to 70% and more preferably from 40% to 60% of amphoteric surfactant; and preferably from 20% to 70% and more preferably from 40% to 60% of anionic surfactants.

Preferably, depending upon the alternative composition, the % of surfactant portion, based on the total weight of the powder composition, is from 20% to 80%; more preferably from 40% to 80%, and most preferably from 50% to 70%.

Heat Generating Agents:

In another component, heat-generating agents or heat generating powders release heat via exothermic reactions (heat producing) with water when they are mixed with water during application. The heat generating agents of the present invention are inorganic salts.

Examples of inorganic salts include, but are not limited to, chloride such as calcium chloride, magnesium chloride, aluminum chloride, ferric chloride, and zinc chloride; sulphates such as magnesium sulphate, zinc sulphate, ferrous sulphate, aluminum sulphate, calcium sulphate, and sodium sulphate; dry alum; calcium oxide; magnesium oxide; sodium carbonate; zeolite; sodium hydrogen phosphate.

Preferred in alternative embodiments are anhydrous inorganic salts such as calcium chloride, magnesium chloride, calcium oxide, magnesium sulphate, sodium sulphate, and aluminum sulphate. An even more preferred optional embodiment includes anhydrous calcium chloride and magnesium sulphate, and mixtures thereof.

The heat-generating agents of the present invention have an average diameter of, preferably from 0.01 um (micron) to 2 mm (millimeter) and preferably from 0.10 um to 500 um.

Since particle size and diameter has a direct effect on potential reactive surface area, particle size has a substantial effect on how fast heat is generated and also the duration of the warming effect. The inorganic salts of larger diameter tend to give a slower but longer warming effect. More preferably, the diameter is from 1 um to 50 um, 80-150 um, 200-250 um and 300-400 um.

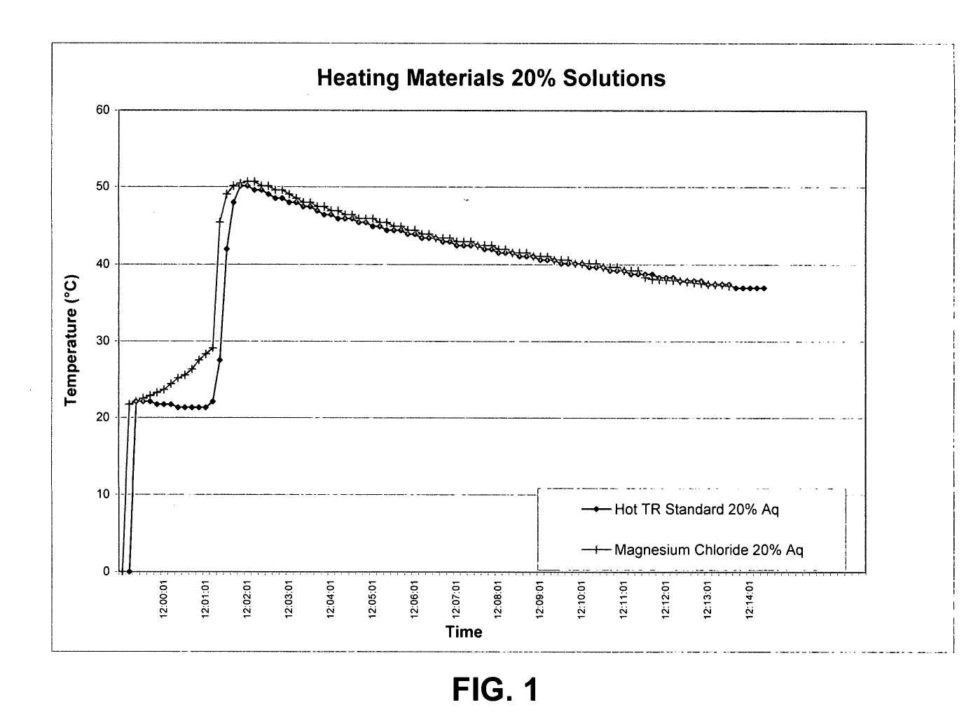

In alternative embodiments, coating the heat-generating particles, with waxes or oils or other agents thickeners, renders particle's surface more lipophilic (an attribute referring to an affinity to fats and oils, or to an ability to solubilize lipids) and will consequently serve to partially delay the onset of exothermic reaction, mitigate the reaction rate, and therefore extend the duration of the warming effect in use (as can be see in FIG. 1, as will be described).

Preferably, the heat-generating agents, based on the total weight of formulation, are from 1% to 80%, and in alternative embodiments more preferably from 10% to 55% (by weight).

Referring now to FIG. 1, a comparison of two (similarly prepared) heating materials, with different heating agents, was conducted. A first heating material Calcium Chloride (Trade Name Hot TR) and a second heating material Magnesium Sulphate, were prepared as described above supporting the desired exothermic reaction and duration of the warming effect. The two heating materials were combined in a 20% aqueous solution. The time, duration, and temperature of each solution was recorded and plotted.

As can be noted from the graph, the initial warming effect was quite steep, from roughly 20 C to about 50 C (which is generally a comfortable user maximum temperature). Additionally noted is the substantial and lengthy cool down slope, changing roughly 12 C in over 12 minutes. This delayed temperature drop is desirable, and as a consequence, it is proposed that compositions made according to the guidelines presented herein will be useful to an end consumer desiring a warming effect employing the compositions disclosed herein.

Skin Benefit Agents:

In another optional and alternative component, a variety of skin benefit agents may be included into the composition, including generally described cosmetic benefit agents and pharmaceutical benefit agents. As used generally herein, cosmetic benefit agents are broadly meant to be any ingredient that is for cosmetically treating, providing nutrients to, and/or conditioning hair and/or skin, via topical application. As generally used herein, a pharmaceutical benefit agent is any hydrophobic or hydrophilic drug that is medically appropriate for topical application.

Examples of skin benefit agents include ingredients selected from these general classes of benefit agents (e.g., descriptions of general classes of skin benefit agents), namely: emollients, anti-aging actives (e.g., agents), anti-wrinkle actives, anti-acne actives, sun screeners, skin lighteners, antibacterial actives, fungicides, anti-diaper rash and skin irritation actives, wound-healing promoters, perfumes, coloring agents, anti-poison ivy/oak/ and anti-itching agents, and mixtures thereof.

The amount of skin benefit agents, based on the total weight of powder composition, is present from about 0.01% to about 30%, preferably from 0.10% to 20%, and more preferably from 0.1% to 15%.

Specific examples of selected skin benefit agents of the present invention include, and may optionally include combinations of: zinc oxide, salicylic acid, benzoyl peroxide, menthol, pathenol, zinc pyrithione, polyquaternium 10, vitamin A and derivatives thereof, vitamin B and derivatives thereof, vitamin E and its derivatives thereof, keratin, lysine, arginine, hydrolyzed protein, silicone oil, retinol, minoxidil, tretinoin, octyl methoxycinnamate, avobenzone, finasteride, camphor, turpentine oil, capsaicin, hydrocortisone, hexylene glycol, lactic acid, mineral oils, and mixtures thereof.

Fillers:

In another optional component, the term “fillers” is generally (but not exclusively) meant to represent solid powdery materials, that generally function to improve the aesthetic and functional properties of the final powder mixtures or as may be used in other composition (e.g., fillers may be used in thick fluids, plastic, and paste forms of the compositions to similarly improve aesthetic and functional properties).

Fillers can also be materials that provide abrasion to the skin to enhance exfoliation. As noted above, in use, fillers may also function to thicken the final composition and may therefore may optionally include selected thickeners noted above. Fillers may be organic, inorganic, or a combination of both. Fillers may be separately machined to a selected particle size prior to introduction into a composition, enabling a selectable particle size distribution.

Examples of inorganic fillers include talc, mica, kaolin, chalk, sericite, muscovite, lepidolite, vermiculite, organically modified magnesium aluminum silicate, organically modified montmorillonite clay, boron nitrite, sodium bicarbonate, and colloidal silicone dioxide. Examples of organic fillers include powders or microspheres of nylon, methyl polymethacrylate, polyethylene, cellulose, styrene/acrylate copolymer, dextrin, and starch, sand, jojoba, cocoa, coffee and other similar fillers.

In selected optional embodiments, fillers are preferably present, based on the total weight of powder composition, from about 0.00% to about 25%, more preferably from about 1.00% to about 20%, and most preferably from about 1.00% to about 10%. As noted above, while fillers are not mandatory, they are useful enhancements to selected compositions for marketability reasons.

Fragrance/Flavors:

In another optional component of the present discussion, a variety of fragrancelflavors/tastes may be optionally included in the composition to provide sensory benefits to a user.

Examples of fragrances/flavors optionally include, but not limited to, nature identical materials such as cocoa, coffee, tea, cucumber, apple, caramel, and where appropriate to an optional selected composition, related man-made fragrance and flavor materials.

As noted above, while fragrances and flavors are not mandatory, they are useful enhancements to selected compositions for marketability reasons. In selected embodiments fragrances/flavors are present from about 0.01% to 10% (by weight), and more preferrably from about 0.10% to about 5.00%. The quantity of fragrance/flavor used may vary depending upon a desired final product.

It is finally noted that minor amounts of known coloring agents (in addition to the other agents listed above) may be alternative included into the composition to achieve a consumer-desirable appearance (e.g., titanium dioxide or other colorents may be used).

The following examples will more fully illustrate selected alternative embodiments of the present invention. All parts, percentage, and proportions referred to herein and in the claims are expressed by weight unless otherwise indicated.

### EXAMPLES 1 to 5

The self-warming compositions were prepared by heating oil/wax combination to 75 C (Celsius), then adding a surfactant and surface coating calcium chloride or magnesium sulphate. The compositions were mixed until they were homogenous. Each composition was evaluated to confirm the desired warming effect by the following protocol. Approximately 5.0 gram of the selected oil/wax/surfactant/heating agent combination placed between two hands, then 5.00 ml of water was added and the two hands were rubbed together activating the composition and discerning the desired warming effect. General warming effects according to the weight % of heat generating agent were confirmed.

TABLE 1

| Components | Example 1 | Example 2 | Example 3 | Example 4 | Example 5 |
|---|---|---|---|---|---|
| Sunflower oil | 43.0% | 32.0% | 44.0% | 45.0% | 36.3% |
| C20-40 Preth-3 | 7.0% | 5.0% | 2.0% | 10.0% | 6.7% |
| Sodium Cocoyl Isethionate | 8.0% | 8.0% | 10.0% | 10.0% | 12.0% |
| Calcium | 35.0% | 50% | 40.0% | 35.0% | 45.0% |
| Polyethylene | 7.0% | 5.0% | 4.0% | — | — |

TABLE 2

| Components | Example 1 | Example 2 | Example 3 | Example 4 | Example 5 |
|---|---|---|---|---|---|
| Sunflower oil | 43.0% | 32.0% | 44.0% | 45.0% | 36.3% |
| C20-40 Preth-3 | 7.0% | 5.0% | 2.0% | 10.0% | 6.7% |
| Sodium Cocoyl Isethionate | 8.0% | 8.0% | 10.0% | 10.0% | 12.0% |
| Magnesium Sulphate | 35.0% | 50% | 40.0% | 35.0% | 45.0% |
| Polyethylene | 7.0% | 5.0 | 4.0 | — | — |

TABLE 3

| Components | Example 1 | Example 2 | Example 3 | Example 4 | Example 5 |
|---|---|---|---|---|---|
| Sunflower oil | 43.0% | 32.0% | 44.0% | 45.0% | 36.3% |
| Polyethylene | 7.0% | 5.0% | 2.0% | 10.0% | 6.7% |
| Sodium Cocoyl Isethionate | 8.0% | 8.0% | 10.0% | 10.0% | 12.0% |
| Magnesium Sulphate | 35.0% | 50% | 40.0% | 35.0% | 45.0% |
| Polyethylene | 7.0% | 5.0 | 4.0 | — | — |

One of the preparation methods discussed herein includes the steps of preparing a selected self-warming compositions by heating a desired oil/wax/thickener combination to approximately 75 C (Celsius), then adding one or more selected surfactants and surface coating prepared heat generating agents having a desired particle size. The heating agent being selected and prepared prior to or after coating to a desired diameter and particle size distribution (normal, bi-modal, etc.) prior to addition of additional elements within an end composition.

The compositions were mixed until they were homogenous. Alternative materials including Fragrances/Flavors, Skin Benefit Agents, Fillers, and coloring agents may be additionally added and similarly mixed during composition to a desired state (dry powder, free flowing liquid, viscous mass, etc.

In alternative embodiments, it is envisioned that additional manufacturing steps may include incorporating the composition into a reusable container (compact, can, jar, tube, etc.), onto a single-use pad for skin application, or into a water resistant bead for storage and use in an aqueous bathroom environment.

In the claims, any means- or step-plus-function clauses are intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the many optional and alternative embodiments of the present invention with reference to the accompanying drawing, it is to be understood that the invention is not limited to those precise embodiments, and that various changes, modifications, adaptations, and alternatives may be effected therein by one skilled in the art without departing from the scope or spirit of the invention as defined in the appended claims.

1. An anhydrous heating skin care composition, comprising:
an exothermic heating component which releases heat when mixed with water during a use;
said heating component being a solid inorganic powder;
said heating component being from about 1% to about 80% by weight of said skin care composition; and
said heating component being coated by a coating component comprising at least one of an oil and a thickener.

2. An anhydrous heating skin care composition, according to claim 1, wherein:
said heating component has a particle size of from about 0.010 microns to about 2.00 millimeters.

3. An anhydrous heating skin care composition, according to claim 1, wherein:
said coating component is from about 0.10% to about 40% by weight of said skin care composition.

4. An anhydrous heating skin care composition, according to claim 1, wherein:
said heating component is an inorganic salt.

5. An anhydrous heating skin care composition, according to claim 1, wherein:
said heating component is at least one of an inorganic salt and an oxide of one of the following metals: sodium, calcium, magnesium, aluminum, iron, lithium, and potassium.

6. An anhydrous heating skin care composition, according to claim 5, wherein:
a conjugate acid of said inorganic salt is at least one of a sulphate, sulfite, chloride, nitrate, and phosphate.

7. An anhydrous heating skin care composition, according to claim 1, wherein:
said thickener is an anhydrous thickener.

8. An anhydrous heating skin care composition, according to claim 1, wherein:
said thickener being at least one of a hydrocarbon based elastomer, a powdered elastomer, a synthetic wax, a naturally occurring wax and a filler.

9. An anhydrous heating skin care composition, according to claim 1, wherein:
said thickener includes at least a wax;
said wax being having a solidus at about 25 degrees C.; and
said wax being at least one of a synthetic wax and a naturally occurring wax.

10. An anhydrous heating skin care composition, according to claim 1, further comprising:
a surfactant;
said surfactant including at least one ionic surfactant portion; and
said ionic surfactant portion including at least one of an anionic and an amphoteric surfactant.

11. An anhydrous heating skin care composition, according to claim 10, wherein:
said surfactant having a liquid-solid phase transformation at about 25 degrees C.

12. An anhydrous heating skin care composition, according to claim 1, further comprising:
a filler component.

13. An anhydrous heating skin care composition, according to claim 12, wherein:
at least one of said thickener and said filler component are selected from a group consisting of a wax, a starch, a clay or modified clay, cellulose or a modified cellulose, an elastomer, silica, an inorganic filler, and an organic filler.

14. An anhydrous heating skin care composition, comprising:
an exothermic material that releases thermal energy when combined with water;
said exothermic material having a particle size of from 0.10 microns to 2.00 millimeters; and
said exothermic material being substantially a solid at room temperature and from about 5% to about 70% by weight of the entire skin care composition.

15. An anhydrous heating skin care composition, comprising:
a heat-generating coated inorganic powder component from 1% to 80% by weight;
at least one of an oil and a thickener component from about 1%-90% by weight; and
at least one surfactant component from about 1%-50% by weight.

16. An anhydrous skin care composition according to claim 15, further comprising:
at least one of a fragrance and a flavor component and a coloring component; and
said at least one being from about 0.01% to 10% by weight of said composition.

17. An anhydrous skin care composition according to claim 15, further comprising:
at least one skin benefit agent component from about 0.01% to about 30% by weight of said composition.

18. An anhydrous skin care composition according to claim 15, further comprising:
at least one filler component from about 0.10% to about 25% by weight of said composition.

19. A skin care composition, comprising:
a heat-generating component including an inorganic salt;
at least one of a oil and a thickener component;
a surfactant component;
a fragrance or flavor component; and
a skin benefit agent component.

20. A skin care composition, comprising:
a heat-generating component including an inorganic powder;
at least one of an oil and a thickener component;
an ionic surfactant component;
at least one of a fragrance and a flavor component; and
a skin benefit agent component.

21. A method for manufacturing an anhydrous heating skin care composition, comprising the steps of:
selecting a suitable heat generating inorganic powder material;
said heat generating material having a particle size range from about 0.10 microns to about 2.00 millimeters;
coating said heat generating powder with at least one of an oil and a thickener; and
intermixing at least one ionic surfactant into said composition.

22. A method for manufacturing, according to claim 21, further comprising the steps of:
incorporating at least one of a skin care benefit agent, a fragrance, a flavor, a filler, and a coloring agent into said skin care composition.

23. A method for manufacturing, according to claim 22, further comprising the steps of:
forming said intermixed skin care composition into one of a powder, a compact solid mass, a liquid, a viscous mass, and a thixotropic suspension.

24. An anhydrous heating skin care composition product made according to the method of claim 21, wherein:
said skin care composition product generates a desirable exothermic reaction when mixed with water, wherein said exothermic reaction enables a raising of a temperature of said skin care composition at least 20 degrees C. following an initial mixing.

25. An anhydrous heating skin care composition product, according to claim 24, wherein:
said composition product is in the form of one of a powder, a compact solid mass, a liquid, a viscous mass, and a thixotropic suspension.

26. A skin care product, comprising:
an anhydrous heating skin care composition made according to claim 22;
a transportation mechanism for said skin care composition whereby a user of said skin care product may easily transport said skin care product and use said skin care composition; and
said transportation mechanism being one of a fibrous cleansing pad, a container bounding a volume containing said skin care composition in a past-power form, and a tube containing a squeezable form of said skin care composition.
